(19) United States
(12) Patent Application Publication
(10) Pub. No.: US 2018/0200105 A1
(43) Pub. Date: Jul. 19, 2018

(54) INTRAOCULAR LENS INSERTER CARTRIDGE WITH A TRAILING HAPTIC PROTECTION STRUCTURE

(71) Applicant: RxSight, Inc., Aliso Viejo, CA (US)

(72) Inventors: Hoang Nguyen, Anaheim, CA (US); Ilya Goldshleger, Ladera Ranch, CA (US)

(73) Assignee: RxSight, Inc., Aliso Viejo, CA (US)

(21) Appl. No.: 15/406,715

(22) Filed: Jan. 14, 2017

(51) Int. Cl. A61F 9/00; A61F 2/16

(52) U.S. Cl. CPC A61F 9/0026; A61F 2/1678; A61F 2/167

(57) ABSTRACT

A cartridge of an intraocular lens (IOL) inserter includes an insertion nozzle, having a distal insertion channel; an IOL-folding stage, having a proximal insertion channel; and a haptic protection structure to protect a trailing haptic of the IOL from damage by a push-rod of the inserter. The haptic protection structure includes a proximal guiding groove in the IOL-folding stage, or a distal guiding groove in the insertion nozzle. The haptic protection structure further includes a trailing-haptic notch, to guide a trailing haptic protruding from the proximal guiding groove; and a trailing-haptic retainer, to secure the trailing haptic out of the proximal insertion channel. An intraocular lens inserter includes an inserter cylinder; a push-rod in the inserter cylinder; a cartridge-receiving insertion tip, to receive a cartridge that includes an insertion nozzle, having a distal insertion channel; an intra-ocular lens-folding stage, having a proximal insertion channel; and a haptic protection structure.

### TECHNICAL FIELD

This invention relates to intraocular lens inserters, and more specifically to haptic protection structures in cartridges for intraocular lens inserters.

### BACKGROUND

The techniques of cataract surgery are experiencing continuous, impressive progress. Subsequent generations of phacoemulsification platforms and newly invented surgical lasers keep increasing the precision of the placement of intraocular lenses (IOLs) and keep reducing the unwanted medical outcomes.

In a typical cataract procedure, an IOL is placed and folded into a cartridge, which is then inserted into a tip of an inserter. Subsequently, the cartridge at the tip of the inserter is inserted into the eye through a surgically created incision, reaching the capsule of the eye. Then the IOL is pushed out of the cartridge through its insertion channel by a push-rod into the eye-capsule, where it is oriented according to the surgical planning, and then stabilized.

IOLs typically have two haptics attached to them. These are thin flexible arms that press against the wall of the capsule after the insertion of the IOL, thereby stabilizing the IOL at the center of the capsule. When the IOL is still in the insertion channel, one of its haptics is typically positioned in front of the IOL, the other behind, trailing the IOL. The push-rod is pushed by the surgeon to force the IOL forward through the same insertion channel where the trailing haptic is positioned. Therefore, in some cases, the push-rod may hit the trailing haptic in the insertion channel, bending and damaging it. Damaged haptics cannot stabilize the IOL in its centered and oriented position. Thus, if the haptic damage is discovered before the insertion of the IOL, then the cartridge with the damaged haptic has to be replaced with a new cartridge with a new IOL. If the damage is discovered only after the insertion, then the surgeon has to remove the deployed damaged IOL from the capsule of the eye with a quite invasive and undesirable procedure, and insert a new one. This takes time and effort, and carries a certain degree of risk. Therefore, there is a profound need for cartridges, which reduce or even eliminate the probability of the push-rod damaging the trailing haptic.

### SUMMARY

The above-described needs are addressed by a cartridge of an intraocular lens inserter, that comprises an insertion nozzle, having a distal insertion channel; an intra-ocular lens (IOL)-folding stage, proximal to the insertion nozzle, having a proximal insertion channel; and a haptic protection structure to protect a trailing haptic of the IOL from damage by a push-rod of the IOL inserter. The haptic protection structure can include a proximal guiding groove, formed in the IOL-folding stage, or a distal guiding groove, formed in the insertion nozzle. The haptic protection structure can further include a trailing-haptic notch, to guide a trailing haptic protruding from one of the proximal guiding groove and the distal guiding groove, out of the proximal insertion channel; and a trailing-haptic retainer, to secure the trailing haptic out of the proximal insertion channel.

Some embodiments include an intraocular lens inserter that comprises an inserter cylinder; a push-rod, at least partially in the inserter cylinder, a cartridge-receiving insertion tip, to receive a cartridge that includes an insertion nozzle, having a distal insertion channel; an intra-ocular lens-folding stage, proximal to the insertion nozzle, having a proximal insertion channel; and a haptic protection structure.

### BRIEF DESCRIPTION OF THE DRAWINGS

FIG. 1 illustrates an inserter 100.

FIG. 2 illustrates an inserter 100 with a cartridge 200.

FIG. 3 illustrates an IOL 10 loaded into a cartridge 200.

FIG. 4 illustrates a perspective view of a cartridge 200 from a proximal front.

FIGS. SA-B illustrate perspective views of a cartridge 200.

FIG. 6 illustrates a longitudinal cross section of a cartridge 200.

FIG. 7 illustrates an open cartridge 200 with an IOL 10 loaded.

FIG. 8 illustrates a cartridge 200 with an IOL 10 during the folding process.

FIGS. 9A-B illustrate a cartridge 200 with a folded IOL 10, pushed by a push-rod 110.

### DETAILED DESCRIPTION

This document describes embodiments of ophthalmic inserters and their cartridges that provide improvements regarding the above described medical needs.

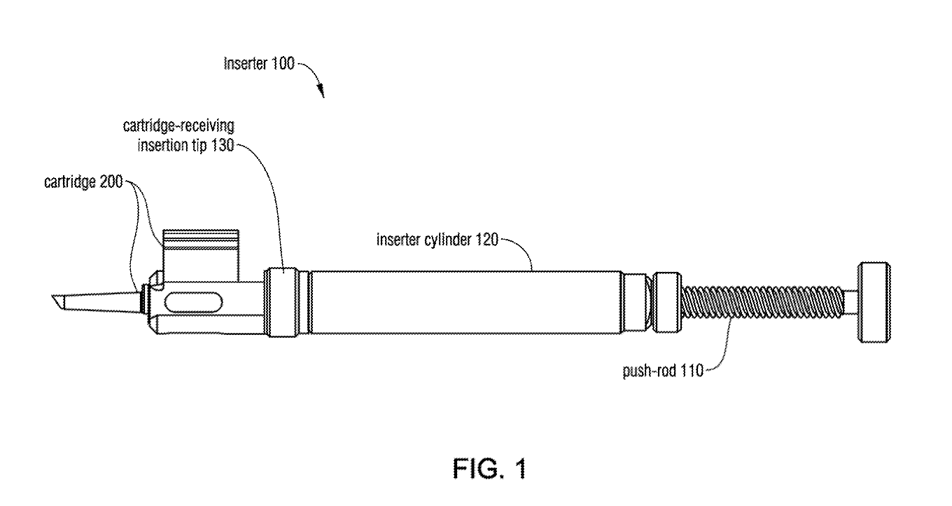

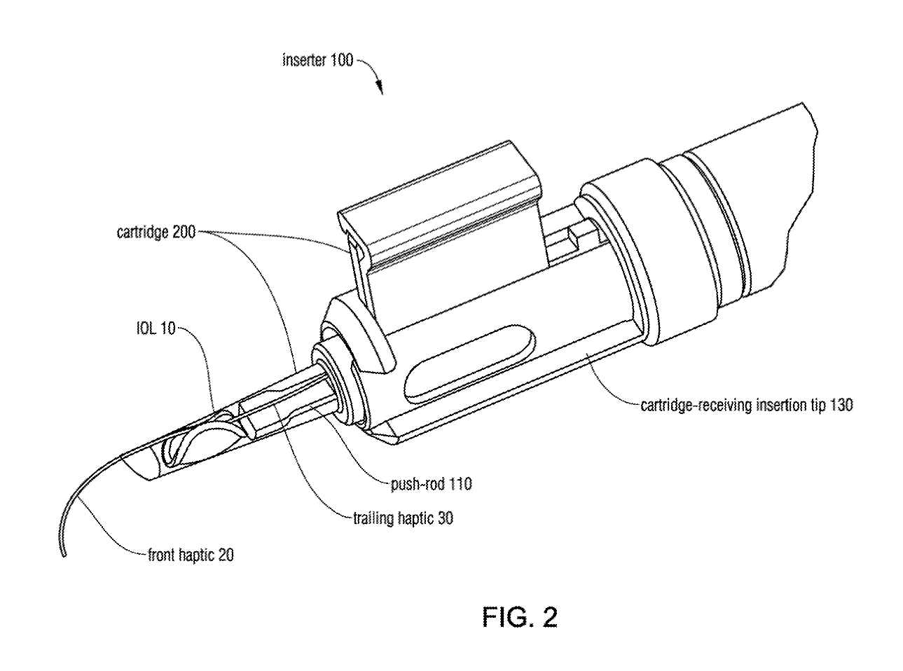

FIG. 1 illustrates an inserter 100 for use in cataract surgeries to insert an intra-ocular lens (IOL) 10 into the capsule of the eye through an incision made by the surgeon. The main components of the inserter 100 include a push-rod 110, an inserter cylinder 120, and a cartridge-receiving tip 130. Some inserters 100 are re-usable, others are use-once disposable devices. A typical operation can include the following steps. (1) An IOL 10 is placed and folded into a cartridge 200. (2) The cartridge 200 is positioned into the cartridge-receiving insertion tip 130 of the inserter 100. (3) The distal tip of the cartridge 200 is inserted into an eye through an incision, created earlier by the surgeon. (4) FIG. 2 shows that the IOL 10 is then pushed forward and eventually inserted from the cartridge 200 into the eye by advancing the push rod 110 forward. The push-rod 110 can be advanced by turning a screw, as shown, or by direct pushing, or by a variety of other known mechanical solutions. In some embodiments, the inserter cylinder 120 can be referred to as a main body, and the cartridge-receiving tip 130 can be referred to as a cover.

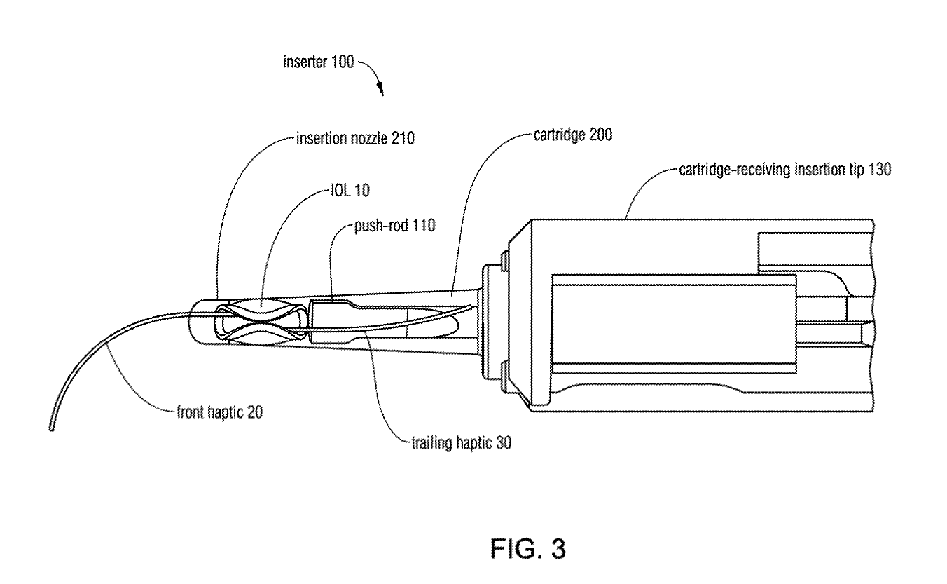

FIGS. 2-3 illustrate that most embodiments of the IOL 10 have two haptics. A front haptic 20 is typically positioned ahead, or distal of the IOL 10 in the cartridge 200, and a trailing haptic 30 is positioned behind, or proximal to the IOL 10. The IOL 10 is being pushed forward into the eye by the push-rod 110 through an insertion nozzle 210. Visibly, the push-rod 110 and the trailing haptic 30 are positioned in the same section of the insertion nozzle 210. In such designs, there is a chance that the push-rod 110 may bend, deform, and possibly break the trailing haptic 30, any of which undermines the medical utility of the IOL 10.

Embodiments of the here-described cartridge 200 are designed to reduce, to minimize and possibly to eliminate the probability of the push-rod 110 damaging the trailing haptic 30.

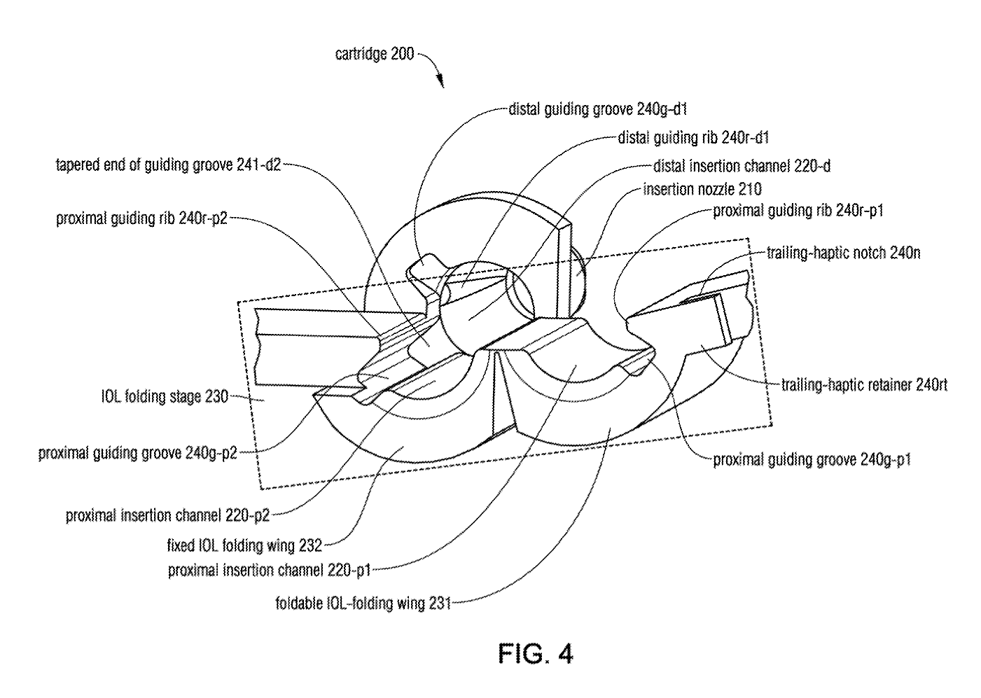

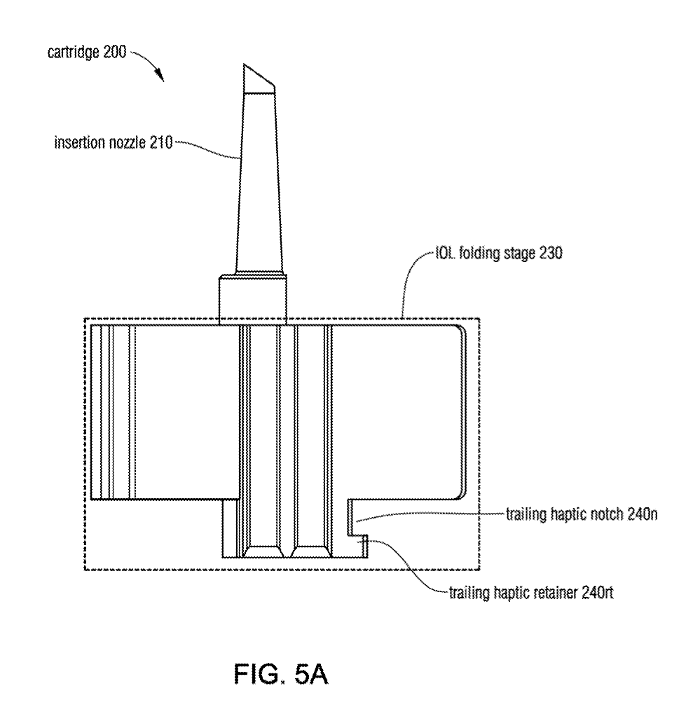

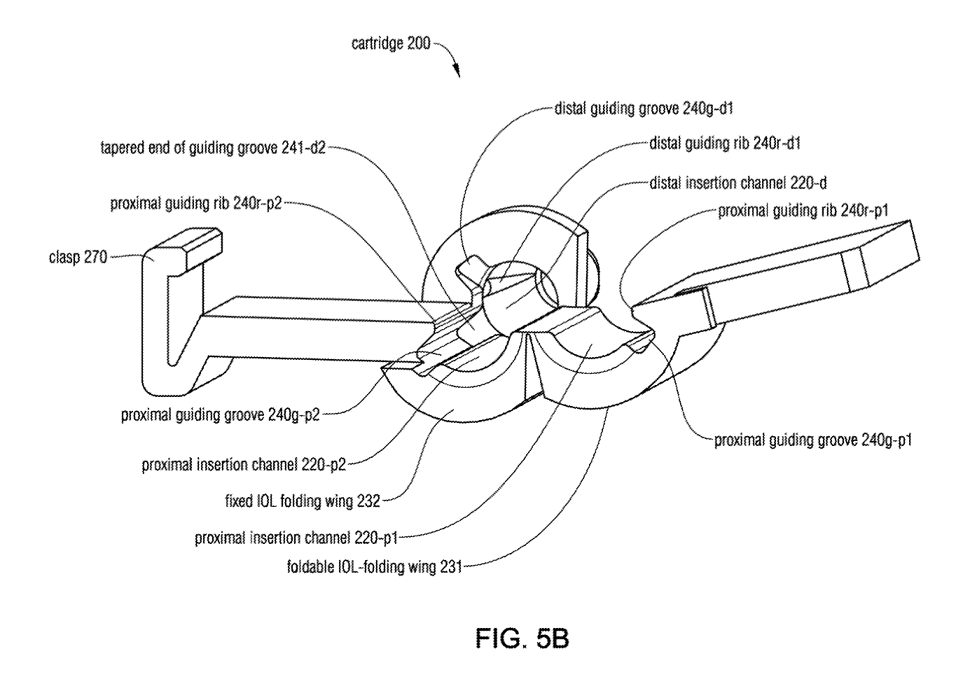

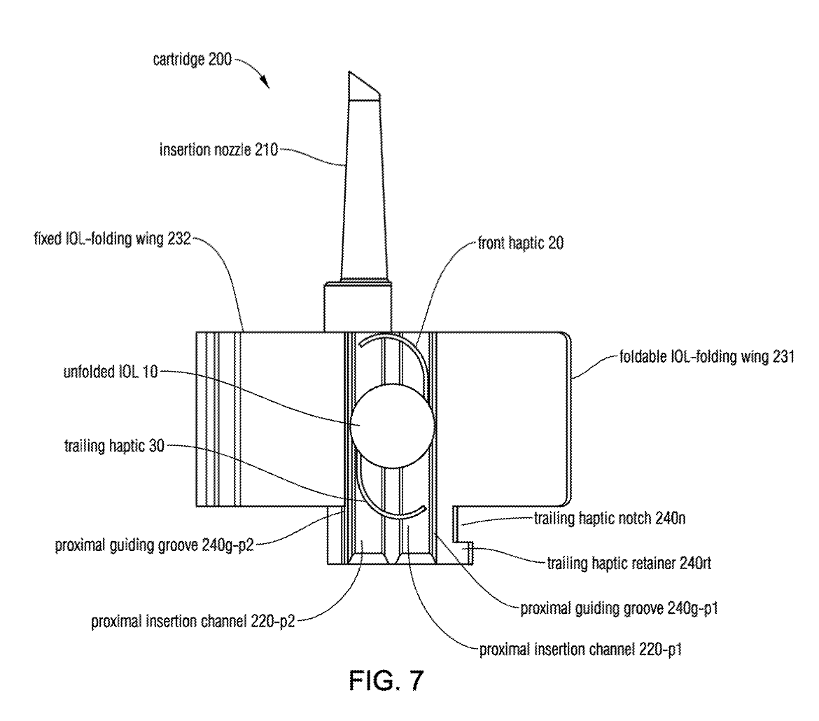

FIGS. 4-9B illustrate embodiments of the cartridge 200 that include an insertion nozzle 210, having a distal insertion channel 220-d; an intraocular ophthalmic lens (IOL)-folding stage 230, proximal to the insertion nozzle 210, having a proximal insertion channel 220-p; and a haptic protection structure 240. In some detail, FIGS. 4-6 describe the structure of embodiments of the cartridge 200 from different perspectives, and FIGS. 7-9B illustrate the positioning of the IOL 10 into the cartridge 200 from different perspectives and at different stages. The below description focuses on the various embodiments and variants of the haptic protection structure 240. In this application, the general convention is adopted that the letters p and d in the labels refer to “proximal” and “distal”, respectively.

In some embodiments, the haptic protection structure 240 can include a proximal guiding groove 240g-p2, formed in the IOL-folding stage 230. This proximal guiding groove 240g-p2 can guide and thus protect the trailing haptic 30 of the IOL 10, as described below in detail.

The haptic protection structure 240 can further include a proximal guiding groove 240g-p1, formed in the IOL folding stage 230, and a distal guiding groove 240g-d1, formed as shown. These guiding grooves can guide the front haptic 20, and can guide the IOL 10 as well. The guiding grooves 240g-d1, 240g-p1 and 240g-p2 together will be referenced as guiding grooves 240g.

The IOL-folding stage 230 can include a foldable IOL-folding wing 231, to partially receive the IOL 10, and a fixed IOL-folding wing 232, to partially receive the IOL 10. In some embodiments, the proximal guiding groove 240g-p1 can be formed in the foldable IOL-folding wing 231, and the proximal guiding groove 240g-p2 can be formed in the fixed IOL-folding wing 232.

FIGS. 4 and 5B show that in some embodiments, the foldable IOL-folding wing 231 and the fixed IOL-folding wing 232 can form the proximal insertion channel 220-p, created by two halves 220-p1 and 220-p2, when the foldable IOL-folding wing 231 is clasped to the fixed IOL-folding wing 232 with a clasp 270.

FIG. 7 illustrates that the loading of the IOL into the cartridge 200 can start with positioning the IOL 10 onto the foldable IOL-folding wing 231 and the fixed IOL-folding wing 232, over, or in, the two halves of the proximal insertion channel 220-p1 and 220-p2.

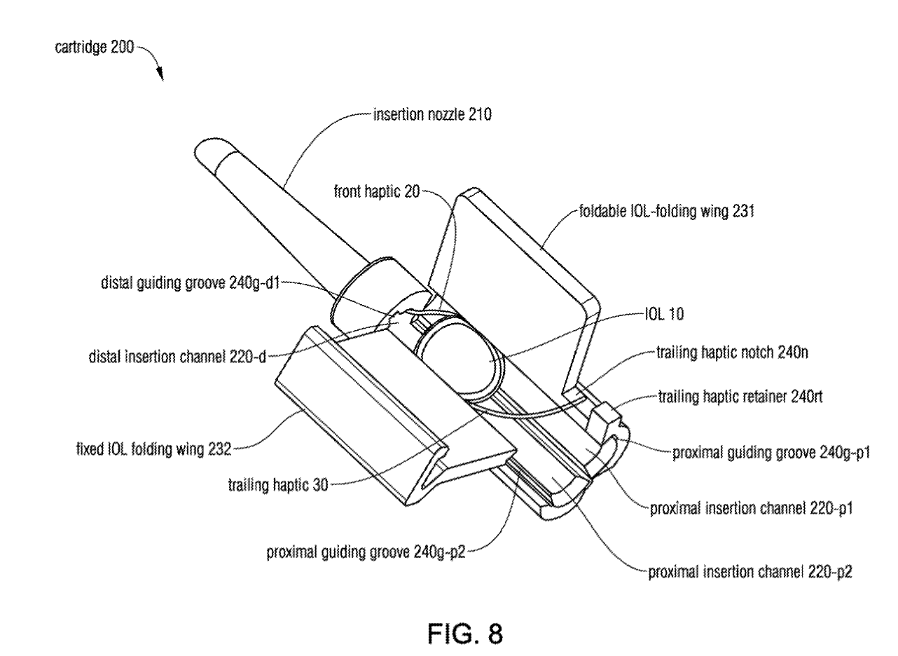

FIG. 8 illustrates that the distal, front haptic 20 can be directed, or positioned into the distal insertion channel 220-d. In typical cases, the front haptic 20 can be positioned into the proximal guiding groove 240g-p1. The proximal guiding groove 240g-p1 becomes aligned with the distal guiding groove 240g-d1 after the foldable IOL wing 231 is folded. Therefore, the front haptic 20 will be able to smoothly glide from the proximal guiding grove 240g-p1 into the distal guiding groove 240g-d1 as the IOL 10 is pushed forward and into the insertion nozzle 210. Guiding the front haptic 20 into these guiding grooves 240g-p1/240g-d1 prevents the bending and possibly breaking of the front haptic 20 in the very tight space of the insertion nozzle 210 during insertion.

Further, also prior to confining and folding the IOL 10, the trailing haptic 30 is guided into the proximal guiding groove 240g-p2, and its proximal end is tucked into the trailing haptic notch 240n. These steps guide the trailing haptic 30 out of the way of the push rod 110, and thus prevent the bending or breaking of the trailing haptic 30 by the push rod 110. After the positioning of the front haptic 20 guiding grooves 240g-p1 and 240g-d1, and the trailing haptic 30 into the proximal guiding groove 240g-p2, the folding of the foldable IOL-folding wing 231 can be carried out, which folds the IOL 10 and confines it into the proximal insertion channel 220-p.

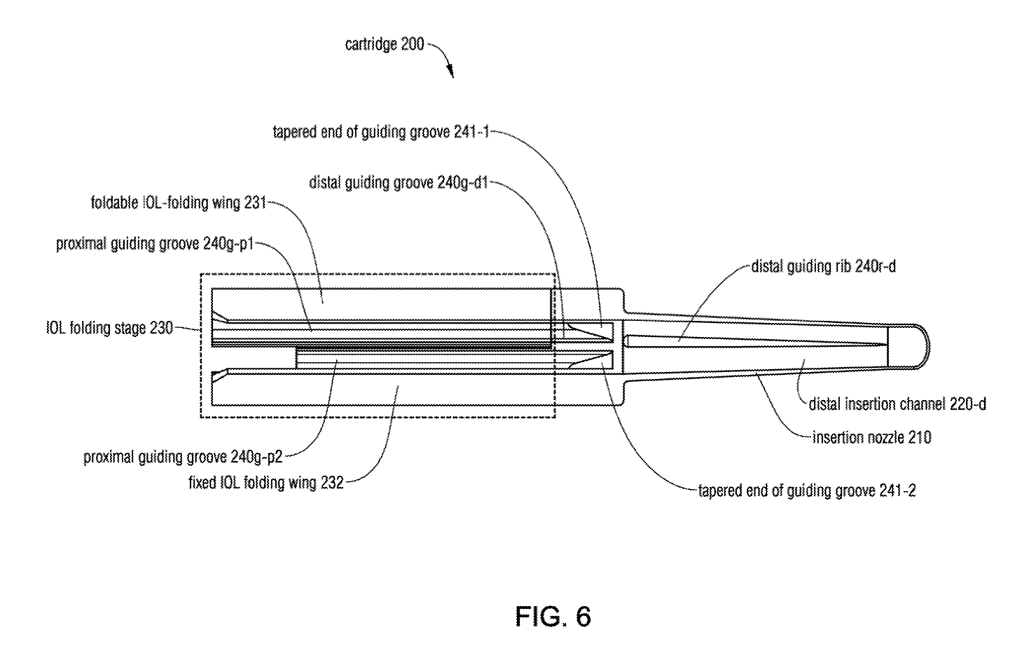

FIG. 6 also illustrates that the guiding grooves can end in a tapered manner, since the entire distal insertion channel 220-d is getting narrower inside the insertion nozzle 210. The aligned guiding grooves 240g-p1/240g-d1 can end with a tapered end 241-1, and the proximal guiding groove 240g-p2 can end with the tapered end 241-2. The proximal guiding groove 240g-p2 can extend beyond the IOL folding stage 230, so that its tapered end 241-2 can align with the tapered end 241-1 of the aligned guiding grooves 240g-p1/240g-d1. One of the reasons for the tapered design is that the insertion nozzle 210 itself can be tapered, its outer diameter decreasing to smaller and smaller values towards its distal tip, so that it can fit into the incision made by the surgeon. In typical cataract surgeries, the incision can be 2.5-3 mm long and accordingly the outer diameter of the distal tip of the insertion nozzle 210 can be reduced to the range of 1-2 mm.

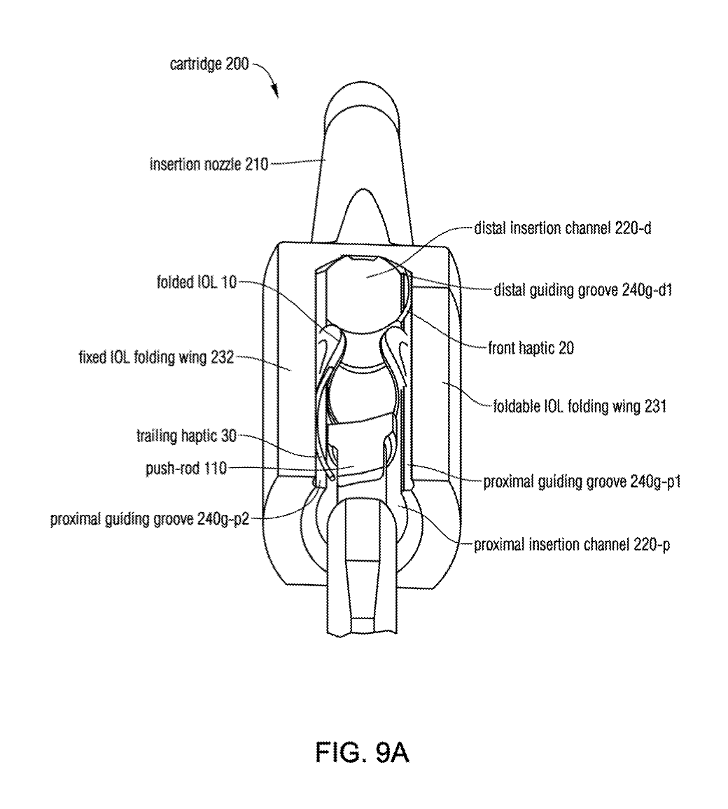

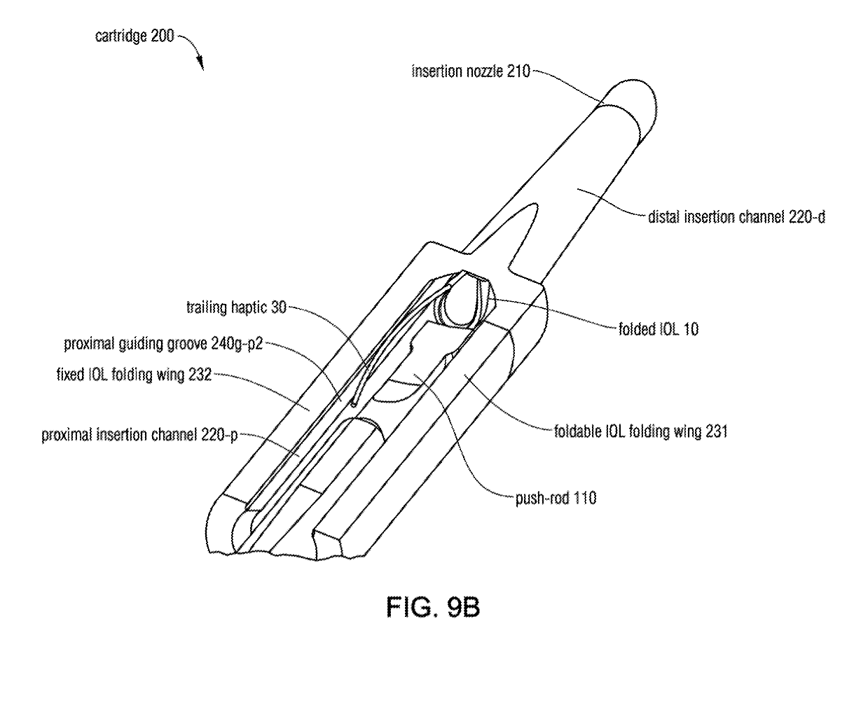

FIG. 9A illustrates a primary function of the haptic protection structure 240. FIG. 9A shows a folded IOL 10 inside the cartridge 200. For clarity, only the inner walls of the cartridge 200 are shown, forming the proximal insertion channel 220-p and the distal insertion channel 220-d. FIG. 9A shows the stage of the IOL insertion as the push-rod 110 is pushing the IOL 10 from the proximal insertion channel 220-p into the distal insertion channel 220-d. Visibly, the push-rod 110 is occupying much of the space in the proximal insertion channel 220-p behind the IOL 10, and therefore would interfere with, and possibly damage, the trailing haptic 30, if the latter were in the same space.

However, embodiments of the cartridge 200 include the haptic protection structure 240 in the form of the proximal guiding grooves 240g-p1 and 240g-p2, and the distal guiding groove 240g-d1. As described earlier, when the IOL 10 is loaded into the IOL folding stage 230, the trailing haptic 30 is positioned, or guided, into the proximal guiding groove 240g-p2, and the front haptic 20 is guided into the proximal guiding groove 240g-p1 and into 240g-d1 by elastic forces, due to the elasticity of the haptic material and the its mechanical design, and by the surgeon, so that it occupies a space physically separate from the push-rod 110. For this reason, the haptic protection structure 240 can reduce, minimize, or eliminate the push-rod 110 damaging the trailing haptic 30, as well as the unwanted bending of the front haptic 20, and thus solves the urgent medical need described in the background section.

FIG. 9A illustrates that after the folding of the foldable IOL folding wing 231, the proximal guiding groove 240g-p1 and the distal guiding groove 240g-d1 are aligned. The front haptic 20 is visibly guided, or positioned, into the proximal guiding groove 240g-p1, seamlessly continuing into the distal guiding groove 240g-d1. Thus, the front haptic 20 is safely positioned and oriented into these guiding grooves during the insertion of the IOL 10, and will avoid getting entangled and possibly damaged in the very tight space of the distal insertion channel 220-d during the insertion.

Also, the trailing haptic 30 is safely guided into the proximal guiding groove 240g-p2, and is clearly positioned outside the path of the push-rod 110 that occupies much of the proximal insertion channels 220-p, and is therefore unlikely to be damaged by the push-rod 110.

FIG. 9B illustrates the same cartridge 200, with the push-rod 110 having moved forward and the IOL 10 having been pushed into the distal insertion channel 220-d. Visibly, the trailing haptic 30 has been guided and positioned in the proximal guiding groove 240g-p2, and kept well-separated from the push rod 110.

As described, the primary function of these guiding groove(s) 240g-p1/240g-d1 and 240g-p2 is (1) haptic protection for the trailing haptic 30, and (2) haptic protection for the front haptic 20. Beyond this, the guiding grooves 240g can have additional functions. (3) The guiding grooves 240g-p1/240g-d1 and 240g-p2 are able to catch corresponding edges of the IOL 10, thereby preventing a rotation of the IOL 10 as it moves along the proximal insertion channel 220-p, and then along the distal insertion channel 220-d during the insertion of the IOL 10. Preventing the rotation of the IOL 10 can be of substantial medical benefit, as for many of the advanced, “patient pay” IOLs, such as for astigmatic and for toric IOLs, the eventual orientation of the IOL in the eye-capsule is key for delivering the planned vision correction. Accordingly, a rotated toric IOL 10 delivers markedly lower vision improvements—a medical outcome to be avoided. Therefore, in some embodiments the grooves 240g-p1/240g-d1 and 240g-p2 can perform both haptic protection and IOL rotation prevention.

(4) Finally, in some embodiments, the proximal guiding grooves 240g-p1 and 240g-p2 can be configured to help folding the IOL 10 by catching an edge of the IOL 10 as part of the folding process. Indeed, in some typical cases, the insertion of the initially flat IOL 10 starts with simply placing the IOL 10 on, or over, the two semi-cylinders of the proximal insertion channel 220-p1 and 220-p2. Then, an operator can start folding the foldable IOL-folding wing 231. Without a mechanical constraint, or coupling, the IOL 10 may pop out, or slide out, from the proximal insertion channels 220-p1 and 220-p2, preventing the controlled folding of the IOL 10. This challenge can be brought under control by the proximal guiding groove 240g-p1, or 240g-p2, or both, catching an edge of the IOL 10, and thus preventing the pop-out, or slide-out, and enabling a well-controlled folding of the IOL 10.

FIG. 6 illustrates that the distal guiding groove 240g-d1 is aligned with the proximal guiding groove 240g-p1. In such embodiments, the caught edge(s) of the IOL 10 can smoothly pass from the proximal guiding groove 240g-p1 to the distal guiding grooves 240g-d1 as the push-rod 110 advances the IOL 10 from the proximal insertion channel 220-p toward the distal insertion channel 220-d.

FIGS. 4 and 5B illustrate that another embodiment of the haptic protection structure 240 can include a distal guiding rib 240r-d1, or proximal guiding ribs 240r-p1 and 240r-p2, referenced in general as guiding ribs 240r. These guiding ribs 240r can function analogously to the guiding grooves 240g. They create a protective space for the front haptic 20 and the trailing haptic 30 that the push-rod 110 cannot enter, thus the trailing haptic 30 can avoid damage by the push rod 110. While the guiding groves 240g create this space for the trailing haptic 30 outside the perimeter of the insertion channels 220, the guiding ribs 240r create the space for the trailing haptic 30 inside the perimeter of the insertion channels 220-p/220-d by pushing the push-rod 110 away from the wall of the insertion channel 220-p/220-d. As shown, in some embodiments of the proximal insertion channel 220-p there can be one proximal guiding rib 240r-p1, in others two: 240r-p1 and 240r-p2.

FIGS. 4-5B, and 8 illustrate another embodiment of the trailing haptic protection structure 240: the trailing-haptic notch 240n and a trailing haptic retainer 240rt. The previously described proximal guiding groove 240g-p2 can guide the trailing haptic 30 away from the push rod 110 inside the insertion channel 220-d/220-p. As an additional layer of protection for the trailing haptic 30, the trailing-haptic notch 240n can guide the trailing haptic 30 out of the proximal insertion channel 220-p after it exist from the proximal guiding groove 240g-p2.

The trailing-haptic retainer 240rt can secure the trailing haptic 30 out of the proximal insertion channel 220-p and thus out of the way of the push-rod 110. After the surgeon places the IOL 10 into the open proximal insertion channel 220-p1 and 220-p2, she can weave the trailing haptic 30 into the trailing haptic notch 240n out of the way of the push-rod 110 which will be pushed through the same proximal insertion channel 220-p after the foldable IOL folding-wing 231 has been folded and the cartridge 200 has been closed. The trailing haptic retainer 240rt, often a protrusion or a bump, can secure the trailing haptic 30 to remain in the trailing haptic notch 240n safely.

Another embodiment can be a functional mirror-image of the above described cartridge 200, wherein the trailing haptic notch 240n and the trailing haptic retainer 240rt are formed in the fixed IOL folding wing 232. Further variant embodiments can be formed by inverting the IOL 10, in which case the proximal guiding groove 240g-p1 would guide the trailing haptic 30. Mirroring, or inverting parts of the system of the cartridge 200 impact its overall functionality, such as the positioning and orienting the IOL 10. Thus, variant embodiments can mirror or invert all corresponding parts of the system of the cartridge 200, but only in configurations that preserve its functionality. This includes the orientation of the IOL 10 as well. If the IOL 10 is positioned in a mirrored or inverted position, it may get inserted into the eye in a backward, or otherwise undesirable position.

All the above embodiments of the haptic protection structure 240, including the guiding grooves 240g-p1/240g-p2 and 240g-d1; the guiding ribs 240r-p/240r-p2 and 240r-d1; the trailing-haptic notch 240n, and the trailing-haptic retainer 240rt can reduce or eliminate the risk of the push-rod 110 bending or damaging the trailing haptic 30 of the IOL, as well as reduce the risk of the front haptic getting entangled or bent. Therefore, in various embodiments, they can be used in any combination towards their shared goal.

While this document contains many specifics, details and numerical ranges, these should not be construed as limitations of the scope of the invention and of the claims, but, rather, as descriptions of features specific to particular embodiments of the invention. Certain features that are described in this document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to another subcombination or a variation of a subcombinations.

1. A cartridge of an intraocular lens inserter, comprising:
an insertion nozzle, having a distal insertion channel;
an intra-ocular lens (IOL)-folding stage, proximal to the insertion nozzle, having a proximal insertion channel; and
a haptic protection structure, to protect a trailing haptic of the IOL from damage by a push-rod of the IOL inserter.

2. The cartridge of claim 1, the haptic protection structure comprising:
a proximal guiding groove, formed in the IOL-folding stage.

3. The cartridge of claim 2, the IOL-folding stage comprising:
a fixed IOL-folding wing; and
a foldable IOL-folding wing, wherein
the proximal guiding groove is formed in one of the fixed IOL-folding wing and the foldable IOL-folding wing.

4. The cartridge of claim 3, wherein:
the fixed IOL-folding wing and the foldable IOL-folding wing form the proximal insertion channel, when the foldable IOL-folding wing is clasped to the fixed IOL-folding wing.

5. The cartridge of claim 2, the haptic protection structure comprising:
a trailing-haptic notch, to guide a trailing haptic out of the proximal insertion channel once it protrudes from the proximal guiding groove; and
a trailing-haptic retainer, to secure the trailing haptic out of the proximal insertion channel.

6. The cartridge of claim 2, comprising:
a second proximal guiding groove; and
a distal guiding groove, aligned with the second proximal guiding groove.

7. The cartridge of claim 6, wherein:
the proximal guiding groove is formed in a fixed IOL folding wing of the IOL-folding stage; and
the second proximal guiding groove is formed in a foldable IOL folding wing of the IOL-folding stage.

8. The cartridge of claim 6, wherein:
at least one the distal guiding groove and the proximal guiding groove has a tapered end.

9. The cartridge of claim 2, wherein:
at least one of
the proximal guiding groove is configured to catch an edge of the IOL, thereby preventing a rotation of the IOL as it moves along the proximal insertion channel during insertion of the IOL; and
the distal guiding groove is configured to catch an edge of the IOL, thereby preventing a rotation of the IOL as it moves along the distal insertion channel during insertion of the IOL.

10. An intraocular lens inserter, comprising:
an inserter cylinder;
a push-rod, at least partially in the inserter cylinder;
a cartridge-receiving insertion tip, to receive a cartridge that includes
an insertion nozzle, having a distal insertion channel;
an intra-ocular lens (IOL)-folding stage, proximal to the insertion nozzle, having a proximal insertion channel; and
a haptic protection structure, to protect a trailing haptic of the IOL from damage by the push-rod of the inserter.

11. The intraocular lens inserter of claim 10, the haptic protection structure comprising:
a proximal guiding groove, formed in the IOL-folding stage.

12. The intraocular lens inserter of claim 10, the haptic protection structure comprising:
a trailing-haptic notch, configured to guide a trailing haptic protruding from one of the proximal guiding groove and the distal guiding groove, out of the proximal insertion channel; and
a trailing-haptic retainer, configured to secure the trailing haptic out of the proximal insertion channel.
